(19) United States
(12) Patent Application Publication
(10) Pub. No.: US 2011/0243924 A1
(43) Pub. Date: Oct. 6, 2011

(54) NOVEL METHODS

(76) Inventor: Jorge Beleta Supervia, Barcelona (ES)

(21) Appl. No.: 12/528,267

(22) PCT Filed: Jan. 31, 2008
(86) PCT No.: PCT/EP2008/000782

§ 371 Date: Aug. 21, 2009

Related U.S. Application Data
(60) Provisional application No. 60/902,843, filed on Feb. 21, 2007.

(51) Int. Cl. A61K 31/439; A61K 31/56; A61K 39/395; A61P 11/00; A61P 11/06; A61P 27/06; A61P 27/02; A61P 25/18; A61P 25/00; A61P 13/08; A61P 1/00; A61P 9/10; A61P 9/00; A61P 25/28; A61P 25/16

(52) U.S. Cl. 424/131.1; 514/305; 514/171

(57) ABSTRACT

The invention provides methods of inhalation treatment of a respiratory disease or condition in a patient in need to such treatment without producing in said patient systemic antimuscarinic effects, comprising administering to said patient an effective amount of aclidinium.

### FIELD OF THE INVENTION

The invention relates to novel methods of anticholinergic therapy, particularly for respiratory diseases such as asthma and chronic obstructive pulmonary disease (COPD), without causing the class-related adverse effects of antimuscarinic compounds.

### BACKGROUND OF THE INVENTION

Aclidinium (3(R)-(2-hydroxy-2,2-dithien-2-ylacetoxy)-1-(3-phenoxypropyl)-1-azoniabicyclo[2.2.2]octane) is a potent muscarinic receptor antagonist described, e.g., in WO 01/04118, WO 05/115467, WO 05/115466, and WO 05/115462 the contents of which applications are incorporated herein by reference. Aclidinium is a long-acting bronchodilator intended for administration by inhalation for treatment of respiratory diseases, especially asthma and COPD), currently in clinical trials.

Currently available muscarinic receptor antagonists include tiotropium ((1α,2β,4β,7β)-7-[(2-hydroxy-2,2-dithienylacetoxy]-9,9-dimethyl-3-oxa-9-azoniatricyclo[3.3.1.0^{2,4}]nonane), ipratropium ([8-methyl-8-(1-methylethyl)-8-azoniabicyclo[3.2.1]oct-3-yl]3-hydroxy-2-phenyl-propanoate), and glycopyrrolate ((1,1-dimethyl-2,3,4,5-tetrahydropyrrol-3-yl) 2-cyclopentyl-2-hydroxy-2-phenyl-acetate).

Acetylcholine is a neurotransmitter associated with parasympathetic innervation in the body and also with transmissions in the brain. It helps control the functioning of the heart, blood vessels, airways, and organs of the urinary and digestive tracts. It is also involved in memory, learning, and concentration. Antimuscarinic compounds inhibit the effects of acetylcholine on muscarinic receptors, which are by far the most common type of cholinergic receptors in the body. Compounds that inhibit acetylcholine activity at the M3 muscarinic receptors in the airways are very useful in the treatment of respiratory diseases, as they inhibit the acetylcholine-mediated contraction of smooth muscle in the airways, resulting in bronchodilation, and also reduce mucus secretion in the lungs.

One problem with the use of antimuscarinic compounds it the treatment of respiratory diseases, however, is the risk of side effects related to systemic suppression of cholinergic activity. These can include, for example, dry mouth, throat irritation, decreased sweating, increased pupil size, blurred vision, increased intraocular pressure, increased heart rate, chest pain, decreased gastric motility, constipation, difficulty starting and continuing to urinate, and loss of bladder control due to overflow incontinence. Anticholinergic activity can also have effects on the central nervous system, such as impaired concentration, confusion, agitation, anxiety, delirium, attention deficit, impaired memory, light-headedness, drowsiness, and respiratory depression. It has been found that cholinesterase inhibitors, which inhibit the breakdown of acetylcholine, are beneficial in

Alzheimer's disease and dementia, thus a physician may wish to avoid anticholinergic drugs in such patients if feasible. Acetylcholine has a complicated role in Parkinson's disease patients. It is believed to have a role in facilitating dopamine release, possibly through actions at the M4 and M5 muscarinic receptors in the brain, and on this basis, cholinesterase inhibitors are sometimes prescribed for Parkinson's patients; yet especially before the advent of levodopa, anticholinergics were used to treat the symptoms of Parkinson's disease, possibly by blocking the dopamine inhibiting activity of the M1 muscarinic receptors.

Older patients are more likely to experience undesired anticholinergic effects because their bodies produce less acetylcholine. Also, cells in many parts of the body (such as the digestive tract) in older patients may have fewer acetylcholine receptors. Thus, the acetylcholine produced is less likely to have an effect, and the effect of anticholinergic drugs is correspondingly greater. Moreover, older patients may have reduced kidney and/or liver function, and so may be prone to increased serum concentrations of many anticholinergic drugs. As discussed below, a number of commonly prescribed medications have anticholinergic effects, so patients who are taking multiple medications with anticholinergic side effects may be at elevated risk. Older men in particular may suffer adverse effects, because the urinary difficulties associated with anticholinergic activity may exacerbate or be exacerbated by an enlarged or obstructed prostate. Overall, anticholinergic side effects are among the most common drug-related negative effects experienced by elderly people.

Currently marketed antimuscarinics may be unsuitable for use in patients having a susceptibility to conditions that may be exacerbated by systemic anticholinergic effects. Levels of systemic anticholinergic activity that may be easily tolerated in a young, healthy person may be unacceptable in such patients. Conditions that may be exacerbated by systemic anticholinergic effects include schizophrenia, glaucoma, dry eyes, enlarged or obstructed prostate, narrowing or obstruction of the small intestine, enlarged colon, chronic constipation, enlarged lower esophagus, heart disease (especially any condition that may be aggravated by tachycardia, for example restenosis or plaque in the coronary arteries, propensity to arrhythmias, damage resulting from prior heart attacks, and congestive heart failure), Parkinson's disease, Alzheimer's disease, dementia, and myasthenia gravis. Antimuscarinics may also present special risks when co-administered with drugs which have anticholinergic effects, for example atypical antipsychotics or tricyclic antidepressants. Antihistamines, particularly first generation sedating antihistamines such as diphenhydramine, may bind muscarinic receptors in addition to histamine type-1 receptors, and so may also have anticholinergic effects. In extreme cases, anticholinergic drugs can trigger anticholinergic delirium, a medical emergency characterized by hot, dry skin, dry mucus membranes, dilated pupils, absent bowel sounds, and tachycardia. Finally, systemically active antimuscarinics may interfere with the action of drugs intended to enhance acetylcholine function, for example cholinesterase inhibitors and cholinergic agonists.

Accordingly, there is a need for antimuscarinic therapy, particularly for respiratory diseases, especially asthma and chronic obstructive pulmonary disease (COPD), which does not cause the class-related adverse effects of systemically active antimuscarinic compounds.

### SUMMARY OF THE INVENTION

It has now been discovered that aclidinium may be used in the treatment of respiratory diseases without exposing patients to the class-related adverse effects of systemically active antimuscarinic compounds. Although aclidinium has the same ester moiety as, e.g., tiotropium (2-hydroxy-2,2-dithien-2-ylacetoxy), aclidinium administered by inhalation is surprisingly much more subject to degradation in plasma to its inactive acid and alcohol metabolites. Consequently, systemic exposure to the compound is negligible. Because of aclidinium's rapid metabolization, it is unlikely to result in undesirable systemic anticholinergic effects. Aclidinium nevertheless has a long duration of action at the receptor and is capable of providing long-acting benefits of antimuscarinic therapy to lungs and airways.

Accordingly, the invention provides, in a first embodiment, the use of aclidinium, in the manufacture of a medicament for use in the treatment or prevention of a respiratory disease or condition in a patient by inhalation, without producing in said patient systemic antimuscarinic effects.

Typically, the respiratory disease is a disease that may be treated, ameliorated or inhibited by a muscarinic receptor antagonist. More preferably, the respiratory disease or condition is selected from acute or chronic bronchitis, emphysema, asthma and chronic obstructive pulmonary disease, especially asthma and chronic obstructive pulmonary disease, most especially chronic obstructive pulmonary disease.

Typically, the patient is suffering from or susceptible to a condition which may be exacerbated by systemic antimuscarinic activity. More typically, the patient is suffering from or susceptible to one or more conditions selected from

a. schizophrenia, impaired concentration, confusion, agitation, delirium, attention deficit, impaired memory, respiratory depression.

b. glaucoma, dry eye, increased pupil size, blurred vision, increased intraocular pressure,

c. enlarged or obstructed prostate, difficulty urinating, overflow incontinence,

d. narrowing or obstruction of the small intestine, enlarged colon, chronic constipation, enlarged lower esophagus, decreased gastric motility, constipation,

e. dry mouth, throat irritation, impaired sweating

f. cardiovascular disease (including any of restenosis, arteriosclerosis, prior stroke or heart attack, congestive heart failure), arrhythmia, tachycardia,

g. Parkinson's disease, Alzheimer's disease, dementia, and/or

h. myasthenia gravis

Typically, the patient is a male. Further, the patient is typically over sixty years old.

In a further embodiment of the invention, the medicament is for administration to a patient who intends to drive or operate machinery during the course of treatment.

In a further embodiment of the invention, the patient is receiving a second drug which is a systemically active anticholinergic agent, or an agent which may cause or exacerbate any of the conditions listed above. Typically, the second drug is selected from antipsychotics, tricyclic antidepressants, and antihistamines.

In a further embodiment of the invention, the patient is receiving a drug which is intended to enhance acetylcholine function, e.g., a cholinesterase inhibitor or cholinergic agonist, e.g., as set forth below.

Typically, the aclidinium is in the form of a salt with an anion X, wherein X is a pharmaceutically acceptable anion of a mono or polyvalent acid. More typically, X is an anion derived from an inorganic acid, such as hydrochloric acid, hydrobromic acid, sulphuric acid and phosphoric acid, or an organic acid such as methanesulphonic acid, acetic acid, fumaric acid, succinic acid, lactic acid, citric acid and maleic acid. Preferably, the aclidinium is in the form of aclidinium bromide.

Typically, the aclidinium is in the form of a dry powder suitable for inhalation.

Typically, the medicament comprises a pharmaceutically acceptable carrier selected from mono-, di- or polysaccharides and sugar alcohols. Preferably, the carrier is lactose.

Typically, the systemic antimuscarinic effect to be avoided is selected from dry mouth, throat irritation, decreased sweating, increased pupil size, blurred vision, increased intraocular pressure, increased heart rate, chest pain, difficulty urinating, enlarged or obstructed prostate, decreased gastric motility, constipation, impaired concentration, confusion, agitation, delirium, attention deficit, impaired memory, and respiratory depression.

Typically, the patient receives one or more additional medication for treatment of the respiratory disease or condition. More typically, the additional medication for treatment of the respiratory disease or condition is selected from beta-adrenergic agonists, corticosteroids or glucocorticoids, PDE IV inhibitors, antihistamines, anti-IgE antibodies, leukotriene D4 inhibitors, inhibitors of egfr-kinase, p38 kinase inhibitors and/or NK1-receptor antagonists; e.g., selected from the compounds identified below. Preferably, the additional medication is selected from corticosteroids and/or beta-adrenergic agonists.

The invention further provides aclidinium, as defined above, or a medicament as defined above, for use in the treatment or prevention, by inhalation, of a respiratory disease or condition, as defined above, in a patient as defined above, without producing in said patient systemic antimuscarinic effects as defined above.

The invention further provides a method of treating or preventing, by inhalation, a respiratory disease or condition as defined above, in a patient in need of such treatment, which patient is as defined above, without producing in said patient systemic antimuscarinic effects as defined above, which method comprises administering to said patient an effective amount of aclidinium, as defined above.

### DETAILED DESCRIPTION OF THE INVENTION

Medications which may have anticholinergic effects or make patients more susceptible to anticholinergic effects, include, for example,

a. Drugs for nausea or dizziness, especially anticholinergic agents, e.g., promethazine (Phenergan), prochlorperazine (Compazine), trimethobenzamide (Tigan), meclizine (Antivert), cyclizine (Marezine), scopalamine

b. Drugs for Parkinson's Disease, especially anticholinergic agents, e.g., benztropine; biperiden; procyclidine; trihexyphenidyl; ethoproprazine

c. Antidepressants, especially tricyclics, e.g., amitriptyline (Elavil), doxepin (Sinequan), imipramine (Tofranil), trimipramine (Surmontil), nortriptyline (Pamelor), protriptyline (Vivactil). amoxapine (Asendin), maprotiline (Ludiomil), clomipramine (Anafranil); desipramine (Norpramin)

d. Antihistamines, especially first-generation sedating antihistamines, e.g., diphenhydramine (Benadryl) chlorpheniramine (Chlor-Trimeton), hydroxyzine (Atarax/Vistaril), cyproheptadine (Periactin)

e. Muscle relaxants, e.g., metaxalone (Skelaxin) cyclobenzaprine (flexeril), orphenadrine (Norflex)

f. Certain anti-migrane medications, e.g., belladonna alkaloids

g. Certain anti-diarrhea drugs, e.g., diphenoxylate/atropine (Lomotil)

h. Urinary and GI Antispasmodics, e.g., oxybutynin (Ditropan), flavoxate (Urispas), dicyclomine (Bentyl), hyoscyamine; belladonna alkaloids; tolterodine (Detrol), trospium, clindinium; propantheline, pirenzepine, telenzepine,

i. Antiarrhythmic drugs, e.g., disopyramide (Norpace), procainamide (Pronestyl), quinidine, atropine

j. Antipsychotics, e.g., chlorpromazine (Thorazine), thioridazine (Mellaril), clozapine (Clozaril), fluphenazine (Stelazine), thiothixene (Navane)

Medications which enhance cholinergic activity, include

a. Reversible cholinesterase inhibitors, e.g., edrophonium, tacrine, donepizil, physostigmine, pyridostigmine, rivastigmine, galantamine, neostigmine,

b. Cholinergic agonists, e.g., methacholine, bethanachol, pilocarpine

Beta-adrenergic agonists that can be combined with aclidinium in the present invention particularly include β2 adrenergic agonists useful for treatment of respiratory diseases or conditions, for example, selected from the group consisting of arformoterol, bambuterol, bitolterol, broxaterol, carbuterol, clenbuterol, dopexamine, fenoterol, formoterol, hexoprenaline, ibuterol, isoprenaline, mabuterol, meluadrine, nolomirole, orciprenaline, pirbuterol, procaterol, reproterol, ritodrine, rimoterol, salbutamol, salmeterol, sibenadet, sulfonterol, terbutaline, tulobuterol, GSK-597901, GSK-159797, KUL-1248, TA-2005 and QAB-1491, in free or pharmaceutically acceptable salt form. Preferably, the β32 adrenergic agonist is a long-acting β2 adrenergic agonist, e.g., selected from the group consisting of formoterol, salmeterol and QAB-149 in free or pharmaceutically acceptable salt form.

Corticosteroids that can be combined with aclidinium in the present invention particularly include those suitable for administration by inhalation in the treatment of respiratory diseases or conditions, e.g., prednisolone, methylprednisolone, dexamethasone, naflocort, deflazacort, halopredone acetate, budesonide, beclomethasone dipropionate, hydrocortisone, triamcinolone acetonide, fluocinolone acetonide, fluocinonide, clocortolone pivalate, methylprednisolone aceponate, dexamethasone palmitoate, tipredane, hydrocortisone aceponate, prednicarbate, alclometasone dipropionate, halometasone, methylprednisolone suleptanate, mometasone furoate, rimexolone, prednisolone farnesylate, ciclesonide, deprodone propionate, fluticasone propionate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, hydrocortisone acetate, hydrocortisone sodium succinate, prednisolone sodium phosphate and hydrocortisone probutate. Budesonide and mometasone are especially preferred.

PDE4 inhibitors that can be combined with aclidinium in the present invention include denbufylline, rolipram, cipamfylline, arofylline, filaminast, piclamilast, mesopram, drotaverine hydrochloride, lirimilast, roflumilast, cilomilast, 6-[2-(3,4-Diethoxyphenypthiazol-4-yl]pyridine-2-carboxylic acid, (R)-(+)-4-[2-(3-Cyclopentyloxy-4-methoxyphenyl)-2-pheraylethyl]pyridine, N-(3,5-Dichloro-4-pyridinyl)-2-[1-(4-fluorobenzyl)-5-hydroxy-1H-indol-3-yl]-2-oxoacetamide, 9-(2-Fluorobenzyl)-N6-methyl-2-(trifluoromethyl)adenine, N-(3,5-Dichloro-4-pyridinyl)-8-methoxyquinoline-5-carboxamide, N-[9-Methyl-4-oxo-1-phenyl-3,4,6,7-tetrahydropyrrolo[3,2,1-jk][1,4]benzodiazepin-3(R)-yl]pyridine-4-carboxamide, 3-[3-(Cyclopentyloxy)-4-methoxybenzyl]-6-(ethylamino)-8-isopropyl-3H-purine hydrochloride, 4-[6,7-Diethoxy-2,3-bis(hydroxymethyl)naphthalen-1-yl]-1-(2-methoxyethyl)pyridin-2(1H)-one, 2-carbomethoxy-4-cyano-4-(3-cyclopropylmethoxy-4-difluroromethoxyphenyl)cyclohexan1-one, cis [4-cyano-4-(3-cyclopropylmethoxy-4-difluoromethoxyphenyl)cyclohexan-1-ol, ONO-6126 (Eur Respir J 2003, 22 (Suppl. 45): Abst 2557) and the compounds claimed in the PCT patent applications number WO03/097613 and PCT/EP03/14722 and in the Spanish patent application number P200302613.

PDE4 antagonists that can be combined with aclidinium in the present invention include tomelukast, Ibudilast, pobilukast, pranlukast hydrate, zafirlukast, ritolukast, verlukast, sulukast, cinalukast, iralukast sodium, montelukast sodium, 4-[4-[3-(4-Acetyl-3-hydroxy-2-propylphenoxy)propylsulfonyl]phenyl]-4-oxobutyric acid, [[5-([[3-(4-Acetyl-3-hydroxy-2-propylphenoxy)propyl]thio]1,3,4-thiadiazol-2-yl]thio]acetic acid, 9-[(4-Acetyl-3-hydroxy-2-n-propylphenoxy)methyl]-3-(1H-tetrazol-5-yl)-4H-pyrido [1,2-a]pyrimidin-4-one, 5-[3-[2-(7-Chloroquinolin-2-yl)vinyl)phenyl)-8-(N,N-dimethylcarbamoyl)-4,6-dithiaoctanoic acid sodium salt; 3-[1-[3-[2-(7-Chloroquinolin-2-yl)vinyl]phenyl]-1-[3-(dimethylamino)-3-oxopropylsulfanyl]methy sulfanyl]propionic acid sodium salt, 6-(2-Cyclohexylethyl)-[1,3,4]thiadiazolo[3,2-a]-1,2,3-triazolo[4,5-d]pyrimidin-9(1H)-one, 4-[6-Acetyl-3-[3-(4-acetyl-3-hydroxy-2-propylphenylthio)propoxy]-2-propylphenoxy]butyric acid, (R)-3-Methoxy-4-(1-methyl-5-N-(2-methyl-4,4,4-trifluorobutyl]carbamoyl]indol-3-ylmethyl]-N-(2-methylphenylsulfonyl)benzamide, (R)-3-[2-Methoxy-4-[N-(2-methylphenylsulfonyl)carbamoyl]benzyl)-1-methyl-N-(4,4,4-trifluoro-2-methylbutyl]indole-5-carboxamide, (+)-4(S)-(4-Carboxyphenylthio)-7-[4-(4-phenoxybutoxy)phenyl]-5(Z)-heptenoic acid and the compounds claimed in the PCT patent application number PCT/EP03/12581.

The words “treatment” and “treating” are to be understood as embracing treatment and/or amelioration of symptoms of a disease or condition as well as treatment of the cause of the disease or condition. Reference to “prevention” of a disease embraces prophylaxis and/or inhibition of the disease.

Aclidinium for use in the methods of the invention may be administered by any suitable route to provide local antimuscarinic action. It is preferably administered by inhalation, e.g., as a powder, spray, or aerosol, preferably as a dry powder. Pharmaceutical compositions comprising aclidinium may be prepared using conventional diluents or excipients and techniques known in the galenic art. For example, dry powder formulations may contain a powder mix for inhalation comprising the aclidinium and a suitable powder base (carrier substance) such as lactose or starch. Use of lactose is preferred. Suitable inhaler devices are known in the art. Dosages will vary depending on, e.g., the individual, the mode and frequency of administration, and the nature and severity of the condition to be treated. Daily dosages for a 40 kg adult human may typically for example be on the order of 100-1000 micrograms of active agent in the form of dry powder for inhalation.

### EXAMPLE 1

### In Vitro Stability of Aclidinium Compared With Tiotropium And Ipratropium And Glycolpyrrolate Stability In Human Plasma

The in vitro experiments are carried out at 36° C. and at a concentration of 5 μg/ml (6 μl of a 1 mg/ml dimethyl sulfoxide solution of each substance is added to a final volume of 1.2 ml). After 3 minutes of pre-incubation, reaction is started by addition of the test substances. At pre-defined times of 0, 5, 15, 30 and 60 min., aliquots of 100 μl of the plasma are separated and the reaction stopped by the addition of 1 ml of a 20 mM, pH 4.0 sodium acetate buffer solution. The test substances are replaced with buffer for the control reactions. Human plasma is obtained from volunteers by written informed consent. The blood is collected in tubes containing lithium heparin as anticoagulant, immediately centrifuged at 4° C. and the resultant plasma stored at −20° C. when not in use.

The determination of aclidinium, tiotropium, ipratropium and glycolpyrrolate in human plasma (100 μl) is carried out by high-performance liquid chromatography (HPLC) using UV detection, at 238 nm for aclidinium and tiotropium, and 203 nm for Ipratropium, and an automated online solid-phase extraction and injection procedure. A suitable chromatographic system consists of a high-pressure pump (model 322 Kontron for aclidinium and tiotropium and model 515 Waters for ipratropium), a Prospekt system (Spark Holland) assisted by a 233XL sampling injector (Gilson Medical Electronics), a tunable absorbance detector (model 2487, Waters Ass.), and a Digital Alpha Server 1000 4/266 computer with Acces*Chrom software (Perkin Elmer Nelson Systems, Inc.). Chromatography for aclidinium and tiotropium determination is carried out on a Spherisorb ODS2, 5 μm, 150×4.6 mm column (Waters Ass.) with a Guardapack gBondapak CN Precolumn (Waters Ass.) and a mobile phase (50:50 v/v for ACLIDINIUM and 22:78 v/v for tiotropium) of acetonitrile: 20 mM, pH 3.0 sodium phosphate buffer solution containing 0.2% triethylamine at a flow rate of 1 ml/min. The approximate retention times for aclidinium and tiotropium are 9.8 and 9.5 minutes respectively. Chromatography for ipratropium determination is carried out on a Symmetry C18, 5 μm, 150×4.6 nun column (Waters Ass.) and a mobile phase (12:88, v/v) of acetonitrile:20 mM, pH 3.0 sodium phosphate buffer solution containing 0.2% triethylamine at a flow rate of 1 ml/min. The approximate retention time of tiotropium is 9.5 minutes. The extraction of aclidinium, tiotropium and ipratropium from plasma is performed on C2 cartridges (Baker) activated with 1.5 ml of acetonitrile and conditioned with 1.5 ml of water. Plasma samples, previously diluted with 1 ml of a 20 mM, pH 4.0 sodium acetate buffer solution, were loaded into the C2 cartridges. After washing out the cartridges with 1 ml of water and 1 ml of acetonitrile:water (40:60, v/v) for aclidinium, or 3 ml of water for tiotropium, or 1 ml of water and 1 ml of acetonitrile:water (10:90, v/v) for ipratropium, the remaining components are eluted with the mobile phase over 1 minute. There are no significant endogenous peaks at retention times of the analytes that would interfere with their quantitation. The recovery of aclidinium is about 95% from human plasma. The recovery of tiotropium and ipratropium from plasma is between 80-100%. Glycopyrrolate stability in human plasma is using essentially the same procedures as for the other three drugs. The lower limit of quantitation is established at 5 mg/ml for all analytes.

Aclidinium is rapidly hydrolyzed in human plasma in its alcohol and acid metabolites. Both metabolites of aclidinium are assayed on binding for M1, M2, M3, and M4 human muscarinic receptors and are devoid of significant affinity for these receptors. The plasma half life of aclidinium in plasma is lower than 5 minutes for human. Moreover, aclidinium is stable in acid aqueous solutions (pH≦4) and the hydrolytic cleavage of the ester bond takes place at neutral and basic pHs.

In contrast, the other three antirnuscarinic esters are quite resistant to degradation by esterases in plasma. Plasma degradation for tiotropium (16%), ipratropium (0%), glycolpyrrolate (9%) is not biologically significant during the time of this study (60 min).

### EXAMPLE 2

Clinical Phase I study: Aclidinium bromide is tested in a Phase I, double-blind, partial cross-over, placebo controlled study to assess the activity, pharmacokinetics and tolerability of aclidinium.

Methods: 12 healthy male volunteers are randomly assigned to 1 of 4 treatment sequences comprising single doses of aclidinium (50, 300 and 600 micrograms) or placebo administered by dry powder inhaler. The washout period between administrations is at least 6 days. Efficacy endpoints are specific airway conductance (sGaw), airway resistance (Raw) and bronchial hyperresponsiveness (PC35 sGaw methacholine).

Results: Aclidinium significantly increases sGaw at all timepoints (1-24 h, p<0.001 vs placebo). Correspondingly, Raw is significantly decreased by aclidinium at all timepoints except 1 h and 24 h (pO.001 vs placebo). Aclidinium 300 and 600 micrograms also significantly reduces PC35 sGaw methacholine at all post-administration timepoints (p<0.001 vs placebo): the methacholine doses required to decrease sGaw by 235% were 142.7 and 181.7 vs 27.1 mg/mL, for aclidinium 300 and 600 micrograms vs placebo, respectively, at 24 h; and 207.1 and 256.0 vs 35.5 mg/mL, respectively, at 2 h. Neither aclidinium nor its metabolites are detected in plasma and no study-drug-related adverse events are reported.

Conclusion: Aclidinium produces significant and long-lasting protection against methacholine-induced bronchoconstriction in healthy male volunteers, demonstrating its suitability for once-daily dosing, notwithstanding that plasma levels are not even detectable.

### EXAMPLE 3

Clinical Phase 11 study: A double-blind, randothised, placebo-controlled, cross-over trial assesses the pharmacodynamics, pharmacokinetics and tolerability of aclidinium and its effects in COPD patients

Methods: Men with COPD (FEV1<65% predicted) with demonstrated airway reversibility to ipratropium are randomised to 1 of 4 treatment sequences comprising single doses of aclidinium (100, 300 and 900 micrograms) and placebo administered by dry powder inhaler with a washout period of 1 week between doses. Lung function measurements include FEV1 and FVC.

Results: 17 males (mean age 63.5 y, mean FEV, 1.63 L) participate in the study. Aclidinium (100, 300 and 900 micrograms) significantly increases mean FEVi AUC(0-24)/24 compared with placebo (1.800 [p=0.002], 1.798 [p<O.OOO1] and 1.827 [pO.OOO1] L vs 1.597 L, respectively). The increase in FEVI are statistically significant at 24 h for all doses. Aclidinium 300 and 900 micrograms produces greater peak FEV1 effects and the time to maximal onset occurres earlier than with the 100 micrograms dose. Similar trends are seen with PVC. No plasma levels of aclidinium or its alcohol metabolite are detected; low levels of its acid metabolite can be detected following the 900 microgram dose. Aclidinium is well tolerated: only 6 cases of mild or moderate headache (vs 2 with placebo) and 1 of mildly increased sweating appear possibly related to treatment.

Conclusion: Single doses of aclidinium (100, 300 and 900 micrograms) produce a rapid and long-lasting bronchodilation in patients with COPD, notwithstanding that plasma levels are not detectable.

1-22. (canceled)

23. A method of inhalation treatment of a respiratory disease or condition in a patient in need to such treatment without producing in said patient systemic antimuscarinic effects, comprising administering to said patient an effective amount of aclidinium.

24. The method of claim 23 wherein the respiratory disease is a disease that may be treated, ameliorated or inhibited by a muscarinic receptor antagonist.

25. A method according to claim 23 wherein the respiratory disease or condition is selected from acute or chronic bronchitis, emphysema, asthma and chronic obstructive pulmonary disease, especially asthma and chronic obstructive pulmonary disease.

26. A method according to claim 23 wherein the patient is susceptible to a condition which may be exacerbated by systemic antimuscarinic activity.

27. A method according to claim 26 wherein the condition which may be exacerbated by systemic antimuscarinic activity is selected from any one or more of:
i. schizophrenia, impaired concentration, confusion, agitation, delirium, attention deficit, impaired memory, respiratory depression,
ii. glaucoma, dry eye, increased pupil size, blurred vision, increased intraocular pressure,
iii. enlarged or obstructed prostate, difficulty urinating,
iv. narrowing or obstruction of the small intestine, enlarged colon, chronic constipation, enlarged lower esophagus, decreased gastric motility, constipation,
v. dry mouth, throat irritation, impaired sweating
vi. cardiovascular disease (including any of restenosis, arteriosclerosis, prior stroke or heart attack, congestive heart failure), arrhythmia, tachycardia,
vii. Parkinson's Disease., Alzheimer's Disease, dementia, and
viii. myasthenia gravis

28. A method according to claim 23 wherein the patient is a male.

29. A method according to claim 23 wherein the patient is over sixty years old.

30. A method according to claim 23 wherein the patient intends to drive or operate machinery during the course of treatment.

31. A method according to claim 23 wherein the patient is receiving a second drug which is a systemically active anticholinergic agent, or an agent which may cause or exacerbate any of the conditions listed claim 27.

32. A method according to claim 31 wherein the second drug is selected from atypical antipsychotics, tricyclic antidepressants, and antihistamines.

33. A method according to claim 23 wherein the patient is receiving a drug which is intended to enhance acetylcholine function.

34. A method according to claim 23 wherein aclidinium is in the form of aclidinium bromide.

35. A method according to claim 23 wherein the aclidinium is in the form of a dry powder suitable for inhalation.

36. A method according to claim 23 wherein the aclidinium is combined with a pharmaceutically acceptable carrier selected from mono-, di- or polysaccharides and sugar alcohols.

37. A method according to claim 36 wherein the carrier is lactose.

38. A method according claim 23 wherein the systemic antimuscarinic effect to be avoided is selected from dry mouth, throat irritation, decreased sweating, increased pupil size, blurred vision, increased intraocular pressure, increased heart rate, chest pain, difficulty urinating, enlarged or obstructed prostate, decreased gastric motility, constipation, impaired concentration, confusion, agitation, delirium, attention deficit, impaired memory, and respiratory depression.

39. A method according claim 23 wherein the patient receives one or more additional medications for treatment of the respiratory disease or condition.

40. A method according to claim 39 wherein the additional medication for treatment of the respiratory disease or condition is selected from beta-adrenergic agonists, corticosteroids or glucocorticoids, PDE IV inhibitors, antihistamines, anti-IgE antibodies, leukotriene D4 inhibitors, inhibitors of egfr-kinase, p38 kinase inhibitors and/or NK1-receptor antagonists.

41. A method according to claim 40 wherein the additional medication is selected from corticosteroids and/or beta-adrenergic agonists.

42. A pharmaceutical composition comprising aclidinium in an amount effective for daily dose to treat a respiratory disease or condition in a patient in accordance with claim 23 without producing systemic antimuscarinic effects in the patient, in combination or association with a pharmaceutically acceptable carrier.
